(19) United States
(12) Patent Application Publication
(10) Pub. No.: US 2019/0262575 A1
(43) Pub. Date: Aug. 29, 2019

(54) CANDLE WAX WARMER AROMATHERAPY DEVICE

(71) Applicant: Saskia de Knegt, Vreeland (NL)

(72) Inventor: Saskia de Knegt, Vreeland (NL)

(21) Appl. No.: 16/287,272

(22) Filed: Feb. 27, 2019

Related U.S. Application Data
(60) Provisional application No. 62/636,037, filed on Feb. 27, 2018.

(51) Int. Cl. A61M 21/02

(52) U.S. Cl. CPC A61M 21/02; A61M 2021/0066

(57) ABSTRACT

A candle device with a support bracket for a melting pot for aroma infused wax and a reusable cuff to extinguish the candle upon implementation is disclosed. The present disclosure teaches an aesthetic, simple and extremely convenient and safe, readily extinguishable dispensing device to dispense volatile active materials, such as air fresheners, fragrances, insect repellants, and mood enhancing aromatherapy compositions into the atmosphere.

This application claims the benefit of U.S. Provisional Application Ser. No. 62/636,037, filed Feb. 27, 2018 and its early file date.

### FIELD OF THE INVENTION

A candle device with a support bracket for a melting pot for aroma infused wax and a reusable cuff to extinguish the candle upon implementation is disclosed. The present disclosure teaches an aesthetic, simple and extremely convenient and safe, readily extinguishable dispensing device to dispense volatile active materials, such as air fresheners, fragrances, insect repellants, and mood enhancing aromatherapy compositions into the atmosphere

### BACKGROUND

Candle and votives are well known. Aroma infused waxes and scented candles are known. Candle devices where a lower candle or votive is used to provide heat to an upper melting dish is also known and commonplace. Placing scented or aroma infused wax in an upper melting dish and allowing candle heat to melt it and allow the aroma to diffuse into the surroundings is also well known. However, these known devices are generally crafted or specially configured to accomplish their task and may be one-piece, or screw together or some other wieldy configuration and not adaptable for use with pre-existing cups or dishes.

In order for a candle to burn, it must have access to a supply of oxygen. A candle burning in an enclosed space without freely circulating oxygen will eventually use up all available oxygen and extinguish itself. Thus, in order for existing candle devices to continue to burn and keep the scented or aroma infused wax melted, there must be an opening in the lower candle burning area.

There have been many attempts at developing new, improved wax melting candle devices. For example, in U.S. Pat. No. 6,780,382 titled SIMMER PLATE DISPENSER FOR VOLATILE ACTIVE MATERIALS, disclosed are, “dispensers for active materials, which dispensers employ heat conductive elements to distribute heat from a burning flame at a wick to a simmer plate and to the body of a solid fuel containing the active material, so as to more rapidly liquefy the solid fuel, such as paraffin wax, and to more uniformly and intensely heat such fuels to volatilize the active material. The heat conductive elements and simmer plate are configured so as to engage said solid fuel, and to cause the flow of liquefied fuel to the wick. The fuel may comprise various active ingredients, such as fragrances, air fresheners, deodorizers, odor eliminators, odor counteractants, insecticides, insect repellants, herbal and medicinal substances, disinfectants, sanitizers, mood enhancers, aroma therapy compositions, and the like, and may be provided in various forms.”

Typically, such devices focus on the efficient melting of the wax, burning of the candle, and diffusing of aroma. Little attention has been given to the simplicity of the design, use of reclaimed materials and/or pre-existing containers and dishes, and importantly, safety and the manner of extinguishing a lit flame.

There have been some attempts at addressing extinguishing the flame. For example, in U.S. Pat. No. 5,944,505 titled, “AUTOMATIC CANDLE SNUFFER, it is disclosed, “a candle holder has candles mounted at each end of a pivoting balance arm. A snuffer is located above each candle. When the balance arm us balanced and one of the candles is lit then the lit candle will become lighter as fuel is consumed by combustion at its wick. When this happens the balance arm pivots so that the end of the balance arm bearing the lit candle rises. This continues until the lit candle is raised to a location at which it is extinguished by one of the snuffers.”

However, most attempts at candle flame extinguishing, are directly related to snuffing out the flame requiring direct access to the flame. Some inventions relate to the wick and adjusting flame size. But again, a requirement is the need to have access to the wick.

Here, disclosed is a simple configuration that provides a minimal clearance between the bottom candle holder and the top wax or other fuel melting dish such that it would be difficult to actually access directly the flame or wick (which is an inherent safety feature). Because the top apparatus will be hot, it would be difficult and dangerous to lift the top wax or other fuel melting dish or the support frame off of the bottom candle holder portion to directly snuff the candle flame. Moreover, because the clearance between the bottom candle holder and top wax or other fuel melting dish is minimal, it would be difficult to fit a traditional candle snuffer into the space to snuff a candle.

An additional element is that once the candle flame is extinguished, hot, molten wax remains for a period of time until the unit cools and the wax solidifies.

None of the foregoing references, alone or in combination, teach the salient and proprietary features or construction of the present disclosure, and as such, fail to be useful as a simplified candle wax warmer with an ease of use and extinguishment with the level of safety provided herein and the ability to use pre-existing containers such as with reclaimed materials.

The present disclosure teaches several embodiments that provide a very convenient, inexpensive, use of recycled or reclaimed materials to craft a simple candle holder with a top wax or other fuel melting dish that is easy and safe to extinguish and containing the bottom candle molten wax from spilling while cooling.

### SUMMARY

The present disclosure teaches embodiments that utilize existing materials, typically reclaimed bottles, configured in such a way as to provide: (i) a bottom candle holder; (ii) a top wax or other fuel melting dish; (iii) a support to support the top wax or other fuel melting dish at an optimal and minimal clearance above the bottom candle holder; and (iv) a cuff to place around the pre-defined clearance depriving the candle flame of needed oxygen for continued combustion and to contain molten wax within the confines of the then sealed bottom candle holder.

In one embodiment, the bottom candle holder can be utilized all by itself as an ordinary candle holder. It is completely independent and usable free from the constraints of the other attachments.

In another embodiment, the bottom candle holder coordinates perfectly with the other components and creates an integrated burn and melt set.

In one embodiment, both the bottom candle holder and top wax or other fuel melting dish are made from identical reclaimed bottles using two bottom pieces cut off from the used containers. In an alternative embodiment, one or both of the bottom candle holder and top wax or other fuel melting dish can be individually crafted. Suitable materials for these items can be chosen from the group of materials including glass, metals, metal alloys, ceramics and any material suitable for resisting cracking, breaking, melting or other deformation upon exposure to a candle flame. In an alternative embodiment, the bottom candle holder and top wax or other fuel melting dish are made from identical reclaimed bottles using two bottom pieces cut off from the used containers and decorated or covered in some manner.

In one embodiment, and typical candle of a size suitable for generally filling the volume of the bottom candle holder can be used. If, as in a preferred embodiment, the reclaimed bottle is a French red wine bottle, such as from a Bordeaux, where there is a substantial indent or impression in the underside bottom of the bottle, the exterior diameter is approximately 2⅞″ and the interior diameter is approximately 2⅝″. In this embodiment, a round candle with a diameter of approximately 2½″ would work well (a typical votive candle). The height of the candle would be determined by the height of bottom candle holder which, in this embodiment would be determined by where a cut of the bottle was made. A typical cut is approximately 3″. However, because of the bottom indent or impression in these types of bottles (which are desired), the candle would sit up approximately 1-1¼″ and leave a space in the bottom of the bottle to accumulate melted wax. One of skill in the art will readily appreciate where to cut a bottle based on the height of the underside impression and the desired votive candle to be implemented.

In a preferred embodiment, as just discussed, the top wax or other fuel melting dish would be created from an identical bottle. However, in creating the top wax or other fuel melting dish, the bottle would get cut at approximately 1½″. When inverted, the indent or impression creates a melting reservoir on the now top portion of the top wax or other fuel melting dish.

### BRIEF DESCRIPTION OF THE DRAWINGS

FIG. 1 depicts one embodiment of the present disclosure wherein one embodiment of a fully assembled and lit device is in operation depicting a bottom candle holder (1) containing a candle is lit wherein the flame (2) meets the bottom underside of a top wax or other fuel melting dish (3) that is supported directly over the bottom candle holder (1) by a support (4) to create a minimal clearance (5) between the top of the bottom candle holder (1) and bottom of top wax or other fuel melting dish (3).

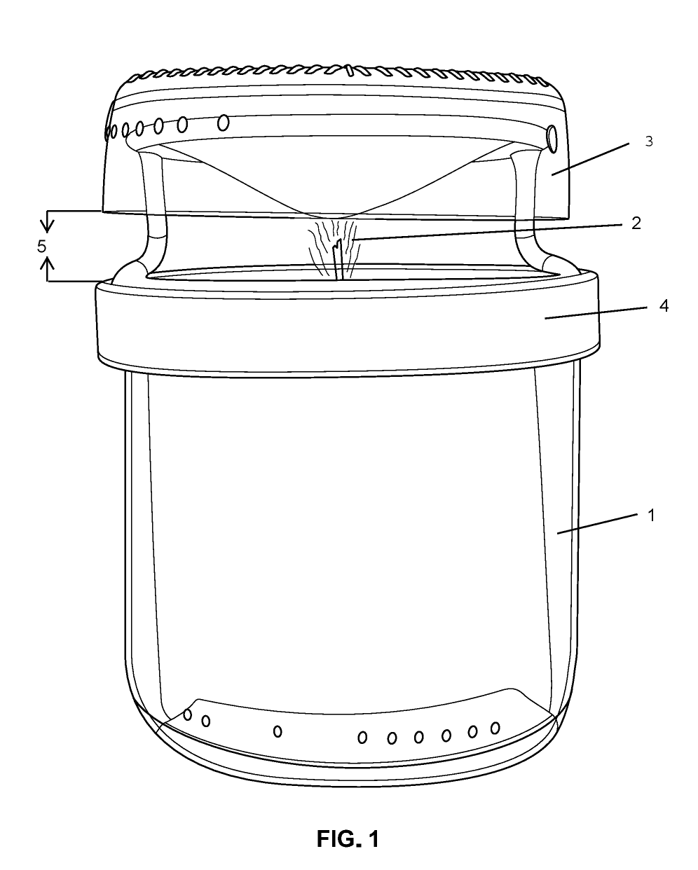

FIG. 2 depicts one embodiment of the present disclosure wherein three of the four salient components are depicted in exploded view in the order in which they are connected, namely, the bottom candle holder (1), the support (4) and the top wax or other fuel melting dish (3).

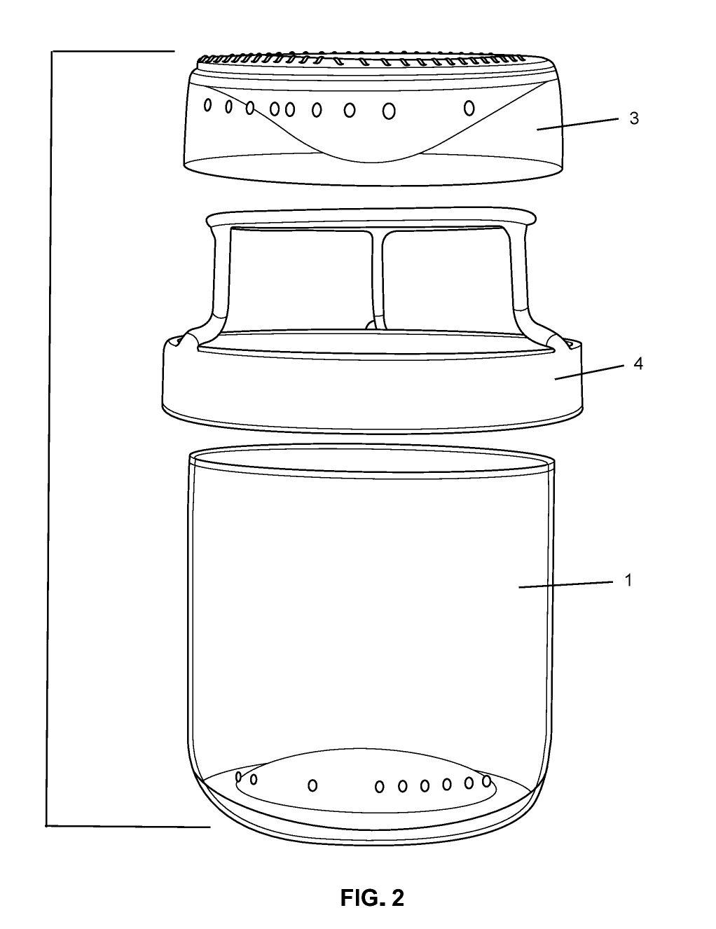

FIG. 3 depicts one embodiment of the present disclosure wherein a representative cuff (6) for use in extinguishing a candle flame and containing molten wax is depicted.

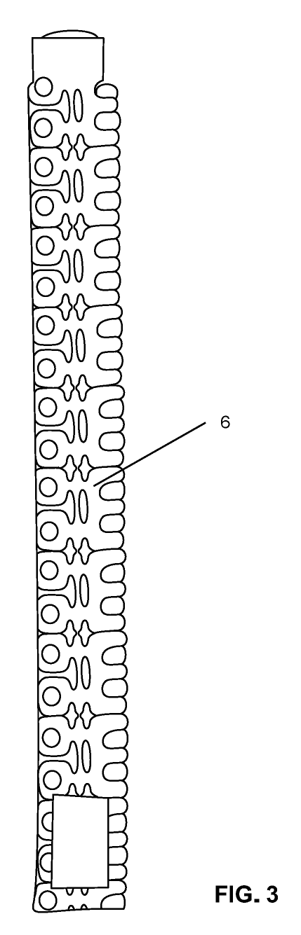

FIG. 4 depicts one embodiment of the present disclosure wherein a typical candle for use with the device as taught herein is shown.

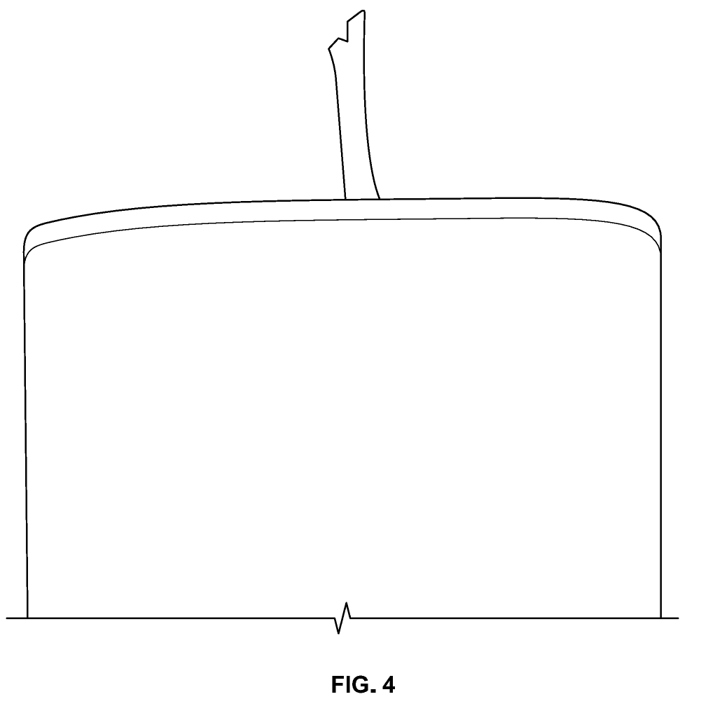

FIG. 5 depicts one embodiment of the present disclosure wherein to use, the candle is placed in the bottom candle holder and is lit.

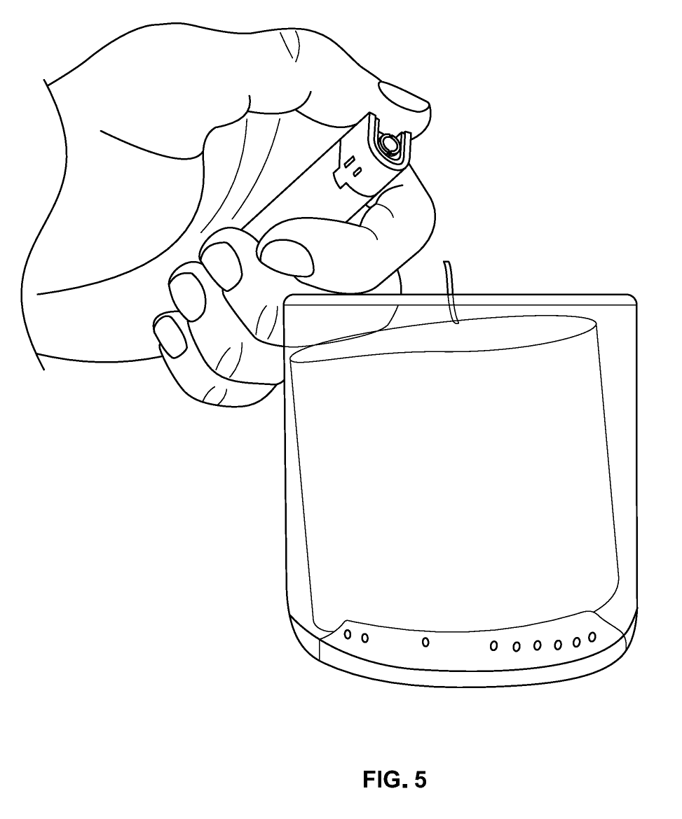

FIG. 6 depicts one embodiment of the present disclosure wherein to further use, once the candle is lit, the support is put into place by setting on the top open edge of the bottom candle holder.

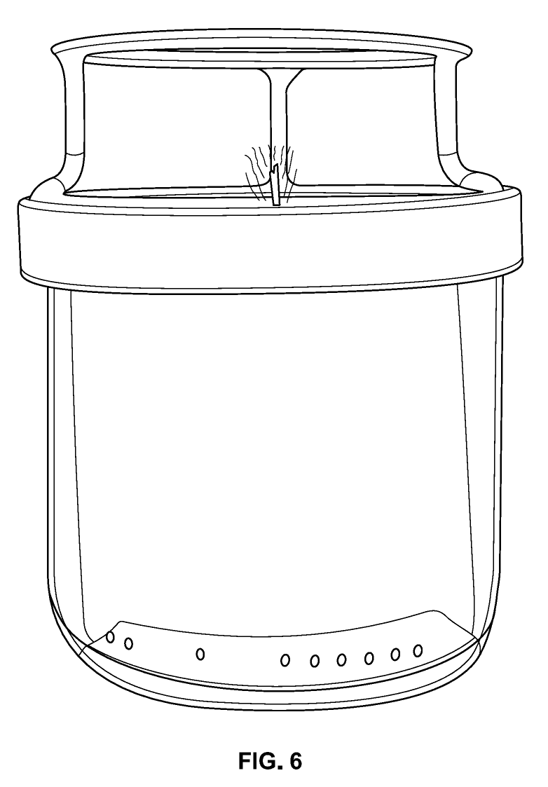

FIG. 7 depicts one embodiment of the present disclosure wherein, continuing use, the top wax or other fuel melting dish is set into place on the support wherein the top of the support is configured to nest into the underside of the open edge of the top wax or other fuel melting dish. Notice that in this embodiment, the bottom of the top wax or other fuel melting dish fits over the support and the inverted open edge comes down the sides so as to create a minimal clearance between the top open edge of the bottom candle holder. In this configuration, there is a minimal supply of oxygen at any given time in the immediate area surrounding the lit flame because the area is very limited.

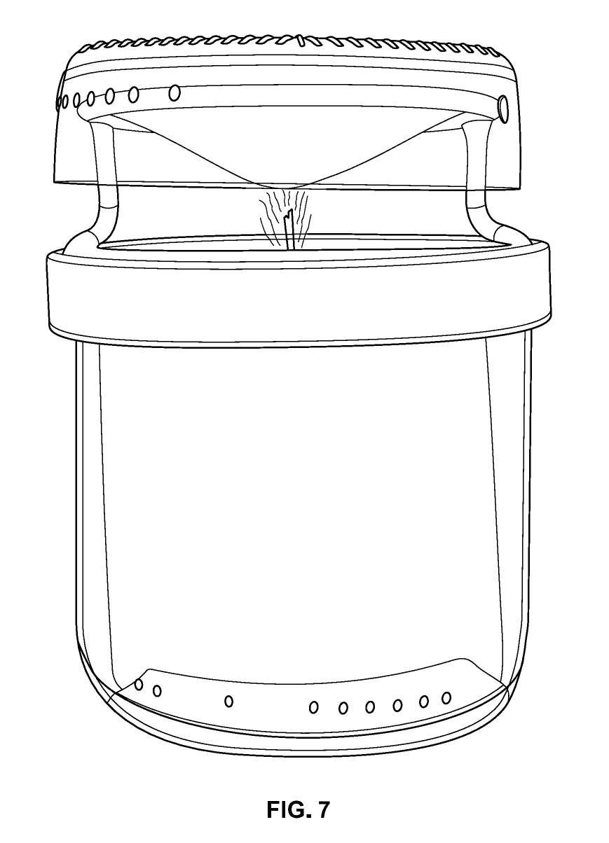

FIG. 8 depicts one embodiment of the present disclosure wherein, continuing use, a special wax or other aromatherapy substance (7) is placed into the top melting dish wherein same will melt from the heat of the under flame and diffuse its infused aroma or other volatile and/or scented substance.

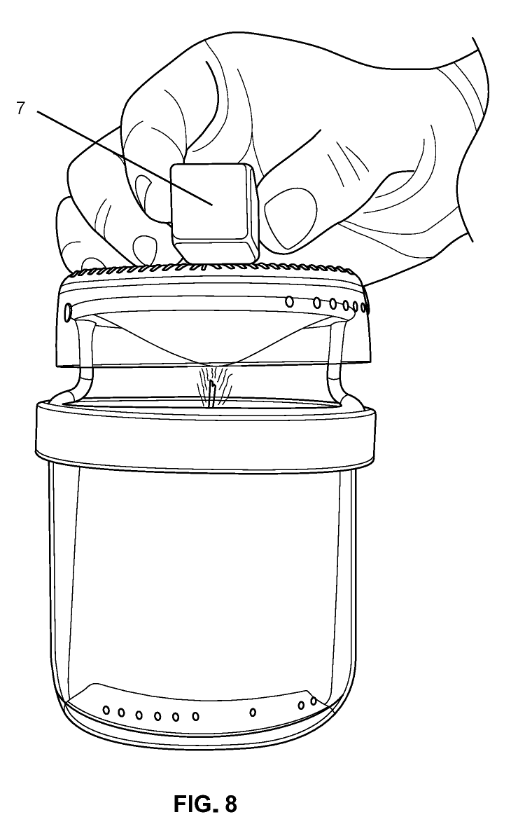

FIG. 9 depicts one embodiment of the present disclosure wherein, when use is desired to be discontinued, a user has no need to directly snuff the candle flame or otherwise disassemble any of the components as assembled as just described. Instead, in this embodiment, the user simply affixes the cuff (6) to cover the minimal clearance between the bottom of the top melting dish and the top of the bottom candle holder as described above and thus quickly extinguishing the flame because there is only a minimal supply of oxygen at any given time in the immediate area surrounding the lit flame because the area is very limited. In one embodiment, the cuff is configured to be of sufficient length to circumnavigate the circumference of the outer edge of the support and overlap and secure via a hook and loop fastener.

### DETAILED DESCRIPTION

For clarity of disclosure, and not by way of limitation, the detailed description of the invention is divided into the following subsections that describe or illustrate certain features, embodiments or applications of the present invention.

### Definitions

“bottom candle holder” as used herein means any suitable container for housing a lit candle preferably with an indent or impression on its bottom surface to raise the candle slightly from the bottom surface having a pre-defined height and circumference.

“top wax or other fuel melting dish” as used herein means a suitable container for melting a wax or other solid fuel with an indent or impression creating a bowl for such use having a pre-defined height and circumference so as to create a minimal gap between the lower surface rim of the dish and the top surface rim of a bottom candle holder when used with a support as described herein.

“support” as used herein means a device with a first ring and lip of pre-defined circumference to nest on top of the top open edge of a bottom candle holder, a second ring of pre-defined circumference to nest in the underside open bottom inside of a top wax or other fuel melting dish and an at least one joining member rigidly connecting the first ring and second ring at a pre-defined distance from each other, preferably configured to be of a height such that when assembled, the resulting gap between the bottom open edge of the top wax or other fuel melting dish and the top open edge of the bottom candle holder is just sufficient to allow a minimal air flow and oxygen to fuel a lit flame, typically approximately ½″. In a preferred embodiment, the support will have three joining members.

“cuff” as used herein means a strip of flexible material having a length sufficient to circumnavigate the entire outer circumference of a bottom candle holder with a support and overlap by approximately ¼-½″ and further having a means to affix the overlapped ends.

### The System and Method of the Present Invention

A typical set-up of one embodiment as described herein is to reclaim two identical bottles each having a bottom with a concave indent or impression. The size of the indent or impression should be more than incidental such as to create a reservoir for melting wax or other fuel when inverted and to keep a candle raised and off the bottom of the candle holder when used as such. Then, cut off each bottle, one at a desired height to function as a candle holder, typically 3″, but could be any height limited only by the height of the bottle; and the second cut off at approximately 1½″, or otherwise as dependent on the depth of the indent or impression. The sides of the bottle should extend just slightly past the depth of the indent or impression. A support is then crafted that is essentially a collar to rest on the upper surface and over the outer edge of the open end of the candle holder, a top ring that is substantially the diameter of the inside diameter of the top wax or other fuel melting dish and rests on the underside of the top surface and fits around the indent or impression and a plurality, typically three, legs that join the bottom collar and top ring and are configured to be of a height such that when assembled, the resulting gap between the bottom of the top wax or other fuel melting dish and the top of the bottom candle holder is just sufficient to allow a minimal air flow and oxygen to fuel a lit flame, typically approximately ½″.

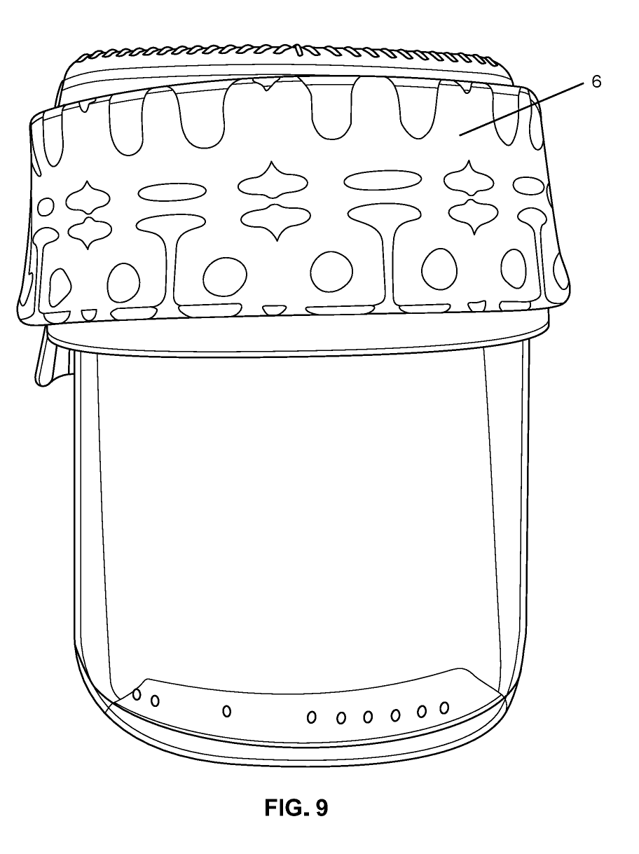

According to one embodiment as described herein and the configuration just described, a cuff, made from any suitable pliable material, typically cotton, is crafted to a length that will cover the entire circumference of the outer surface of the bottom candle holder and overlap slightly, approximately ½″ and be of a width that is larger than the opening created by the resulting gap between the bottom of the top wax or other fuel melting dish and the top of the bottom candle holder, typically 1″. In this embodiment, and substantially as demonstrated in FIG. 9, when the candle is desired to be extinguished, the cuff is wrapped around the gap opening and carefully overlapped and secured, typically with a hook and loop fastener means. In this manner, oxygen will no longer be able to fuel the candle flame and it will naturally extinguish without any need to directly snuff it. The cuff will also seal the gap, hold the support in place and even in the event that the entire device tipped over, only the wax or other fuel in the top melting reservoir will escape, not the candle wax, nor the igniting flame.

In one embodiment, the device as described herein is reusable an infinite number of times, simply by replacing the bottom candle and/or the top wax or other fuel to be melted. By choosing the bottle(s) to be used, varying colors, shapes and sizes can reflect a distinct mood, conjure a specific memory and welcome all who enter the fragranced area of a lit device.

### Examples

The present invention is further illustrated, but not limited by, the following examples.

A typical reclamation of materials is two wine bottles and some iron for the support. A refill candle will typically be of beeswax and Glycine Soja (Soy) Wax. The top wax or other fuel is typically beeswax, Glycine Sojo (Soy) Wax and Perfume or fragrance. The cuff is typically of organic cotton.

In this embodiment, the two wine bottles will be cut such that each bottom is cut off, each at pre-defined heights, one to serve as the bottom candle holder, and the other to serve as the top wax or other fuel melting dish. In this embodiment, each will be of similar diameter and the support is then fashioned as described above and shown in the Figures. A typical candle votive is then placed in the bottom candle holder, lit and the support and top wax or other fuel melting dish are nested in place. An aromatherapy wax is placed in the top dish and the candle is allowed to burn until the user decides to extinguish with the cuff.

In a preferred embodiment, the candle refill will burn for about fifty hours before a refill is needed and the top wax or other fuel will release its fragrance for approximately six hours of use.

Publications cited throughout this document are hereby incorporated by reference in their entirety. Although the various aspects of the invention have been illustrated above by reference to examples and preferred embodiments, it will be appreciated that the scope of the invention is defined not by the foregoing description but by the following claims properly construed under principles of patent law.

Each and every feature described herein, and each and every combination of two or more of such features, is included within the scope of the present invention provided that the features included in such a combination are not mutually exclusive.

What is claimed is:

1. A device for convenient candle burning, wax melting and/or aromatherapy substance heating and lighting and extinguishing comprising:
a bottom candle holder comprising a container with an open top and a top edge;
a top wax or other fuel melting dish comprising a top side compression bowl and an underside open bottom with a bottom edge;
a support comprising:
a first ring and lip of pre-defined circumference to nest on top of the top edge of the bottom candle holder; and
a second ring of pre-defined circumference to nest in the underside open bottom of the top wax or other fuel melting dish; and
at least one joining member rigidly connecting the first ring to the second ring; and
configured to have the first ring set on top of the open top edge of the bottom candle holder and support the top wax or other fuel melting dish via the second ring supporting the underside open bottom of the top wax or other fuel melting dish creating a minimal separation between the bottom edge of the top wax or other fuel melting dish and the top edge of the bottom candle holder covered by the first ring and lip.

2. The device of claim 1 having three joining members rigidly connecting the first ring to the second ring.

3. The device of claim 1 further comprising a cuff, sufficient to circumnavigate the outer circumference of the candle holder fitted with a support and overlap and a means to secure the overlapped ends.

4. The device of claim 1 wherein the bottom candle holder and top wax or other fuel melting dish are substantially round and of substantially the same circumference.

5. The device of claim 4 wherein the bottom candle holder and top wax or other fuel melting dish are made from recycled bottles.
